(19) United States
(12) Patent Application Publication
(10) Pub. No.: US 2024/0082331 A1
(43) Pub. Date: Mar. 14, 2024

(54) ANTI-OBESITY COMPOSITION CONTAINING, AS ACTIVE INGREDIENT, MORINGA OLEIFERA FERMENTATION PRODUCT PREPARED USING NOVEL MONASCUS PURPUREUS SL1

(71) Applicant: MBIOMETHERAPEUTICS CO., LTD., Suwon-si, Gyeonggi-do (KR)

(72) Inventors: Se Chan KANG, Yongin-si, Gyeonggi-do (KR); Jeong Eun KWON, Suwon-si, Gyeonggi-do (KR); Yeong Geun LEE, Suwon-si, Gyeonggi-do (KR)

(73) Assignee: MBIOMETHERAPEUTICS CO., LTD., Suwon-si, Gyeonggi-do (KR)

(21) Appl. No.: 18/270,214

(22) PCT Filed: Nov. 9, 2021
(86) PCT No.: PCT/KR2021/016220

§ 371 Date: Jun. 29, 2023

(30) Foreign Application Priority Data
Jan. 7, 2021 (KR) 10-2021-0001753

(51) Int. Cl. A61K 36/185; A61K 31/351; A61K 31/352; A61K 31/7048; A61P 3/04; C12N 1/14

(52) U.S. Cl. CPC A61K 36/185; A61K 31/351; A61K 31/352; A61K 31/7048; A61P 3/04; C12N 1/145; A61K 2236/19; C12R 2001/645

(57) ABSTRACT

The present invention relates to a composition for controlling anti-obesity activity, containing, as an active ingredient, a Moringa oleifera fermentation product prepared using a novel Monascus purpureus SL1 strain, wherein the Moringa oleifera fermentation product provided in the present invention remarkably inhibits the differentiation of adipocytes and the Moringa oleifera fermentation product of the present invention has anti-obesity effects that are remarkably superior to those of Moringa oleifera powder.

### FIELD OF THE INVENTION

The present invention relates to an anti-obesity composition containing fermented Moringa oleifera extract produced using a novel Monascus purpureus SL1 strain as an active ingredient.

### BACKGROUND OF THE INVENTION

Obesity is characterized by chronic inflammation, which is caused by complex interaction of genetic, metabolic, environmental and behavioral factors. Further, obesity is defined by abnormal or excessive fat accumulation. Such obesity is known to be a significant factor causing to adult diseases such as hypertension, type 2 diabetes, cancer, gallbladder disease, hyperlipidemia, and atherosclerosis. The known causes of obesity indicate that genetic factors account for more than 70%, while the remaining causes are unbalance between energy intake and energy expenditure.

Fat stored in adipocytes is used as an important energy source in the body. However, as obesity progresses, the number of adipocytes increase but synthesis of large amounts of triglycerides by excessive adipocyte differentiation leads to morphological changes of adipocytes including increased size of adipocyte and alterations in gene expression. The increase in adipocyte size is induced by synthesis and storage of excess energy as triglycerides, and adipocyte size can increase up to approximately 20 times according to the storage of fat. While adipocytes size can generally be regulated by diet control, the differentiation of new precursor cells into adipocytes is not significantly affected by diet control only. Therefore, it is important to regulate the process of adipocyte differentiation for the ultimate treatment or inhibition of obesity.

Additionally, adipocytokines, which are biologically active molecules secreted from adipose tissue or adipocytes, play a key role in appetite regulation, insulin sensitivity, energy metabolism and control of the microenvironment of blood vessels. Adipocytokines such as TNF-α, IL-6, MCP-1, and adiponectin are closely related to obesity-induced inflammation and the occurrence of obesity-related diseases. Dysregulation of adipocytokine secretion can induce the chronic inflammation observed in obesity and promote the occurrence of obesity-related diseases.

Currently, anti-obesity drugs include Xenical inhibiting fat digestion and absorption, and Fluoxetine and Phentermine suppressing appetite, and Liraglutide promoting metabolism. However, the mentioned drugs have limited efficacy and come with side effects including cardiovascular, respiratory and neurological disorders. Therefore, it is required to develop anti-obesity drugs that are exceptionally effective and have minimal side effects.

Moringa (Moringa oleifera) is a perennial plant scattered to tropical and subtropical regions. It is widely utilized for various purposes including medicinal uses, cosmetics, water purification, animal feed and crop growth enhancers according to the characteristics of each part of the plant. All parts of Moringa such as leaves, seeds, flowers, stem, fruits and roots, are used as ingredients in food. Moringa leaves, in particular, have been reported to have relatively low moisture content compared to other plant leaves and have a high amount of various nutrients relatively. Moringa leaves have a high amount of protein and minerals including beta-carotene, vitamins, carotenes, calcium, potassium, and iron.

Monascaceae in the present invention is a red-colored mold that grows on fermented foods such as soybean paste, soy sauce and rice wine, and it is called Honggukgyun (Monascaceae) due to its red appearance. It is traditionally used as a food ingredient in various Asian countries including Korea, China, Japan, Hong Kong and Taiwan.

Monascaceae has been utilized as a natural dye to produce a red color as well as food ingredients, and it has a higher alcohol production capacity compared to other yeast strains, and it possesses a strong aroma and excellent flavor. Therefore, it has been used as a natural pigment or preservative in various food products. Additionally, it has the ability to produce various hydrolytic enzymes and exhibits antimicrobial effects.

### DETAILED DESCRIPTION OF THE INVENTION

### Problems to be Solved

The present invention aims to provide a pharmaceutical composition for preventing or treating obesity, containing fermented Moringa (Moringa oleifera) extract produced using a novel Monascus purpureus SL1 strain as an active ingredient.

Additionally, the present invention aims to provide food composition or health functional food composition for preventing or improving obesity.

### Solution

In order to solve the problem, the present invention provides a pharmaceutical composition for preventing or treating obesity, containing fermented Moringa (Moringa oleifera) extract produced using a novel Monascus purpureus SL1 strain as an active ingredient.

Additionally, the present invention provides food composition or health functional food composition for preventing or improving obesity.

Moreover, the present invention provides a method comprising administering the pharmaceutical composition for preventing or treating obesity to a subject.

### Benefits of the Invention

The composition according to the present invention, which is isolated from fermented Moringa extract, Moringa powder and fermentation product thereof inhibits adipocyte differentiation significantly. The composition according to the present invention has exceptional efficacy compared to Moringa powder. Therefore, fermented Moringa extract according to the present invention can be beneficially utilized as a composition for treatment, prevention or improvement of obesity by inhibiting adipocyte differentiation.

### BRIEF DESCRIPTION OF DRAWINGS

FIG. 1 depicts the measurement result of compounds isolated from fermented Moringa extract and Moringa powder.

FIG. 2 depicts the measurement result of adipocyte differentiation rate of fermented Moringa extract and Moringa powder.

FIG. 3 depicts the measurement result of adipocyte differentiation rate of compounds isolated from fermented Moringa extract and Moringa powder.

FIG. 4 depicts the measurement result of adipocyte differentiation rate after liver metabolism of Fermented Moringa extract and Moringa powder.

### BEST MODE FOR IMPLEMENTING THE INVENTION

Hereinafter, the present invention will be described in details.

The inventors of the present invention have confirmed that the fermented Moringa extract and active ingredients isolated from the fermented Moringa extract not only exhibit anti-adipogenic activity but also maintain the efficacy even through hepatic metabolism. Based on the research, the inventors have completed the present invention. The fermented Moringa extract, which is included as an active ingredient in the pharmaceutical composition or health food composition of the present invention, is a natural substance derived from natural raw materials, and has the advantage of extremely low concern for side effects.

The present invention provides a pharmaceutical composition for preventing or treating obesity, comprising fermented Moringa (Moringa oleifera) extract as an active ingredient.

The term “Moringa oleifera” used in the present invention refers to a tree belonging to the Moringaceae family, which grows to a height of 5 to 10 meters and is cultivated in Asia, Africa, Arabia, and other regions. Moringa oleifera is known for high nutritional value, being rich in proteins and vitamins. Moringa oleifera also possesses various pharmacological effects, such as anti-hyperglycemic, anti-inflammatory, and anticancer properties. The flowers, roots, seeds, leaves, and fruits of Moringa are known to contain abundant phytochemicals such as vitamins, flavonoids, and amino acids compared to other plants.

Furthermore, the fermented Moringa extract can be obtained by adding strains of Monascus genus to Moringa powder and fermenting. Preferably, the fermented Moringa extract can be obtained by fermenting with Monascus purpureus, and more preferably with Monascus purpureus SL-1 (Deposit Number KCCM12830P), but not limited thereto.

Additionally, the Moringa powder can be prepared by freeze-drying Moringa leaves and then grinding them into a powder, but not limited to this method.

Moreover, the Moringa powder can be mixed with water at a ratio of 3 to 10 times the weight of the dried Moringa powder, or preferably 8 to 10 times the weight of the dried Moringa powder, and then can be added to the culture medium for seed fungus.

Additionally, the fermentation can be performed at 30 to 40° C., 50 to 200 rpm for 1 to 5 days. Preferably, at 35 to 39° C., 90 to 110 rpm for 1 to 3 days. More preferably, at 37° C., 100 rpm for 2 days, but not limited thereto.

Moreover, the fermented Moringa extract can be obtained by sterilizing at 80 to 100° C. for 1 to 3 hours and spray-drying after fermentation is complete.

Additionally, the fermented Moringa extract can contain one or more selected from a group consisting of niazirin, kaempferol, 6″-O-acetyl astragalin and quercetin as an active ingredient, but not limited thereto.

Additionally, the composition can inhibit the differentiation of adipocyte.

Furthermore, the present invention provides a method for preventing or treating obesity, comprising administering the pharmaceutical composition for preventing or treating obesity, to a subject.

The pharmaceutical composition of the present invention is administered in a pharmaceutically effective amount. The term “pharmaceutically effective amount” used in the present invention refers to a quantity that is sufficient to provide a reasonable ratio of benefit to risk for the medical treatment of a disease, without causing significant side effects. The effective dosage level can be determined based on factors such as the patient's health condition, type and severity of the disease, drug activity, sensitivity to the drug, administration method, administration time, administration route and elimination rate, treatment duration, co-administration with other drugs, and other well-known factors in the medical field. The composition of the present invention can be administered as a single therapeutic agent or in combination with other therapeutic agents. It can be administered sequentially or simultaneously with conventional treatments. It can be administered in a single or multiple doses. Considering all of the above factors, it is important to administer the composition in the minimal amount that achieves maximum effect without causing side effects, and it can be determined by a person skilled in the art. In the present invention, the term “therapeutically effective amount” refers to a pharmaceutically acceptable amount of the composition that is effective in preventing or treating the target disease. The therapeutically effective amount of the composition of the present invention can depend on various factors such as administration method, target site and patient's condition. Therefore, when determining the dosage for administration to human body, the amount should be determined based on safety and efficacy. It is also possible to estimate the amount for use in humans based on the effective dose determined based on animal experiments. Considerations for determining the effective amount are described, for example, in Hardman and Limbird, eds., Goodman and Gilman's The Pharmacological Basis of Therapeutics, 10th ed. (2001), Pergamon Press; and E. W. Martin ed., Remington's Pharmaceutical Sciences, 18th ed. (1990), Mack Publishing Co.

In the present invention, the term “subject” refers to any organism aimed at preventing or treating obesity, and is not specifically limited to humans. It includes animals including non-human primates (such as monkeys, for example cynomolgus monkeys and chimpanzees) as well as non-primate mammals (such as cows, pigs, horses, cats, dogs, rats, and mice) among others. In some cases, it can exclude humans.

The composition according to the present invention can include a pharmaceutically effective amount of fermented Moringa extract alone or in combination with one or more pharmaceutically acceptable carriers, excipients or diluents. The term “pharmaceutically effective amount” refers to an amount sufficient to prevent, improve or treat symptoms of immune-related diseases. The term “pharmaceutically acceptable” refers to a composition that is physiologically tolerated and, when administered to humans, does not typically cause adverse reactions such as gastrointestinal disorders and allergic reactions.

Further, the composition including pharmaceutically acceptable carriers can take various oral or non-oral dosage forms. When formulated, it can be prepared using commonly used fillers, diluents, binders, lubricants, disintegrants, surfactants, or other excipients. The carriers, excipients and diluents can be one or more selected from a group consisting of lactose, dextrose, sucrose, sorbitol, mannitol, xylitol, erythritol, maltitol, starch, acacia gum, alginates, gelatin, calcium phosphate, calcium silicate, cellulose, methyl cellulose, amorphous cellulose, polyvinylpyrrolidone, physiological saline, methyl hydroxybenzoate, propyl hydroxybenzoate, talc, magnesium stearate, mineral oil, dextrin, calcium carbonate, propylene glycol, and liquid paraffin, but not limited thereto. Other commonly used carriers, excipients or diluents can also be used. The ingredients can be added independently or in combination with the active ingredients, the fermented Moringa extract.

Additionally, a solid form for oral administration can include tablets, powders, granules or capsules. The solid forms can be prepared by mixing one or more compounds with at least one excipient such as starch, calcium carbonate, sucrose or lactose, and optionally including lubricants such as magnesium stearate or talc. For a liquid form for oral administration can include suspensions, emulsions, syrups or solutions, which can contain various excipients in addition to simple diluents like water or liquid paraffin, such as humectants, sweeteners, flavoring agents, preservatives and others. For non-oral administration, the formulations can include sterile solutions, non-aqueous vehicles, suspensions, emulsions, lyophilized formulations or suppositories. Non-aqueous vehicles or suspending agents such as propylene glycol, polyethylene glycol, vegetable oils like olive oil and injectable esters like ethyl oleate can be used. Suppository bases such as Witepsol, Macrogol, Tween 61, cacao oil, laurin, glycerogelatin and others can be used as the base for suppository formulations.

Additionally, the pharmaceutical composition of the present invention can have any one of the aforementioned forms selected from a group consisting of tablets, powders, granules, capsules, solutions, suspensions, emulsions, syrups, sterile solutions, non-aqueous vehicles, lyophilized formulations and suppositories.

The effective dosage of the fermented Moringa extract in the human body can depend on factors such as the patient's age, body weight, gender, administration form, health condition and severity of the condition. Generally, it is typically around 0.001-100 mg/kg/day, and preferably 0.01-35 mg/kg/day. Taking a adult patient with 70 kg as a reference, the typical dosage can be around 0.07-7000 mg/day, and preferably 0.7-2500 mg/day. Depending on the physician's or pharmacist's judgment, it can be administered once daily or divided into multiple doses at regular intervals.

Moreover, the present invention provides food product or health functional food composition for preventing or improving obesity or metabolic and inflammatory diseases, comprising fermented Moringa (Moringa oleifera) extract as an active ingredient.

The fermented Moringa extract contained in the food or health functional food composition of the present invention as an active ingredient is the same as described in the present invention. Therefore, the detailed description of the fermented Moringa extract applies equally to the food or health functional food composition of the present invention. To avoid unnecessary repetition and excessive complexity in the specification, common details are omitted.

The food composition of the present invention can be formulated in various forms such as powders, extracts, granules, capsules, liquid preparations and beverages, etc., and thereby can be added to food. There are no specific limitations on the types of food. The fermented Moringa extract of the present invention can be formulated in various forms such as powders, extracts, granules, capsules, liquid preparations, beverages, etc., and can be added to food. Examples of foods in which the fermented Moringa extract can be added include drinks, meat products, sausages, bread, biscuits, rice cakes, chocolates, candies, snacks, cookies, pizza, instant noodles, other noodle products, chewing gum, ice cream, dairy products, various soups, soft drinks, alcoholic beverages, vitamin complexes, dairy products, and processed dairy products. It comprises both general health foods and functional foods with the general view.

The health food and functional food compositions containing the fermented Moringa extract according to the present invention can be directly added to food or used in combination with other foods or food ingredients, and can be used appropriately according to conventional methods. The amount of the fermented Moringa extract can be determined suitably depending on intended use (prevention or improvement). Generally, the amount of the composition in the health food and functional food can range from 0.1 to 90 parts by weight of the total food weight. However, in the case of long-term consumption for the purpose of maintaining health or regulating health, the amount can be lower than the mentioned range, and the active ingredient can be used in amounts exceeding the mentioned range as long as there are no safety concerns.

The composition of health food and functional food formulation according to the present invention can contain, in addition to the fermented Moringa extract of the present invention as essential ingredients, other ingredients without any specific limitations. The composition can include various flavoring agents or natural carbohydrates, similar to common beverages. Examples of the mentioned natural carbohydrates include monosaccharides such as glucose and fructose; disaccharides such as maltose and sucrose; and polysaccharides such as dextrin and cyclodextrin, as well as sugar alcohols like xylitol, sorbitol, and erythritol. In addition to the mentioned flavoring agents, natural flavoring agents (such as thaumatin, stevia extracts like Rebaudioside A, glycyrrhizin, etc.) and synthetic flavoring agents (such as saccharin, aspartame, etc.) can be preferentially used. The ratio of the mentioned natural carbohydrates is generally about 1 to 20 g per 100 g of the health functional food composition according to the present invention, preferably about 5 to 12 g.

In addition to the above mentioned composition, the health food and functional food formulations containing the fermented Moringa extract of the present invention can include various additives such as nutritional supplements, vitamins, minerals (electrolytes), synthetic flavor enhancers, natural flavor enhancers, coloring agents, and stabilizers (used in cheese, chocolate, etc.), pectin and salts thereof, alginic acid and salts thereof, organic acids, protective colloids, thickening agents, pH regulators, stabilizers, preservatives, glycerin, alcohol, carbonating agents used in carbonated beverages, etc.

Furthermore, the health food and functional food compositions of the present invention can include pulp for the production of natural fruit juices, fruit juice drinks, and vegetable beverages.

The ingredients can be used independently or in combination. The proportions of the additives are not critical, but it is common to select them in the range of approximately 0.1 to 20 parts by weight per 100 parts by weight of the health food and functional food composition containing the fermented Moringa extract of the present invention.

### EXAMPLES FOR IMPLEMENTING THE PRESENT INVENTION

Hereinafter, the present invention will be described in details based on examples with referring to drawing. However, the examples are only for helping understand the present invention and the present invention is not limited thereto.

### Example 1. Preparation of Fermented Moringa Extract

Moringa (Moringa oleifera) leaves were freeze-dried and powdered and thereby Moringa powder was produced.

Subsequently, 100 g of the obtained Moringa powder was mixed with 1000 g of water, and Monascus purpureus SL-1 culture medium was added. The mixture was then subjected to reciprocating shaking cultivation at 37° C. for 2 days under the conditions of 100 rpm. The Monascus culture medium was added at a ratio of 5% weight based on the weight of the extract. The Monascus culture medium was concentrated under reduced pressure, and then sterilized again at 95° C. for 2 hours. Finally, the mixture was spray-dried and thus fermented Moringa extract powder was obtained.

### Example 2. Isolation of Compounds from Moringa Powder and Fermented Moringa Extract

100 g, respectively, of Moringa powder and fermented Moringa extract were extracted four time repeatedly at room temperature using 70% EtOH (500 mL×3) for 24 hours. Each extract was concentrated under reduced pressure, yielding 21 g. Among them, 20 g was dissolved in 200 mL of distilled water and then partitioned three times with 200 mL of EtOAc. The water layer was further partitioned three times with 160 mL of n-BuOH. The obtained fractions were concentrated under reduced pressure. Each fraction of Moringa powder and fermented Moringa extract were analyzed by TLC, and significant differences in EtOH fraction was identified.

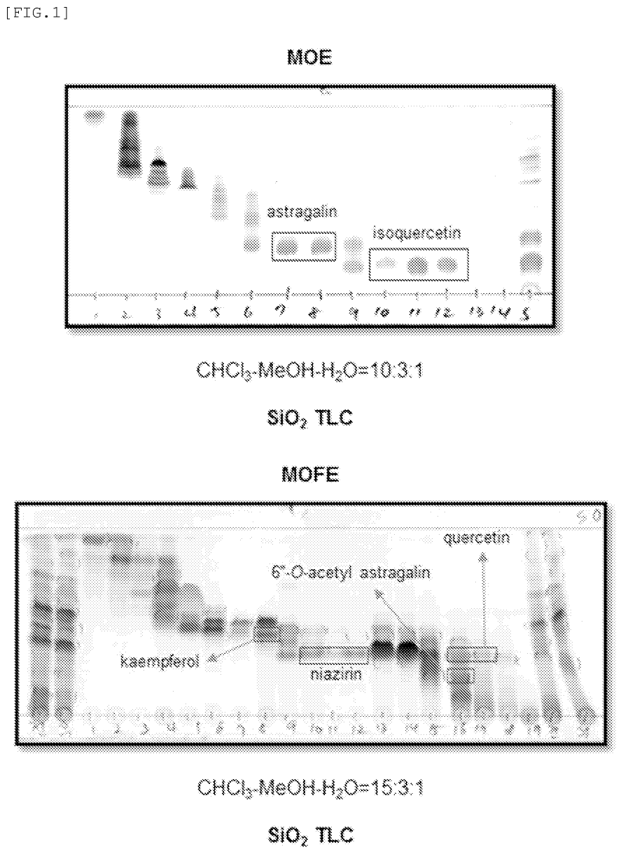

SiO₂ column chromatography was performed on 5 g of EtOH fraction of the Moringa powder. The column with size (φ 5×15 cm) was used, and 10 L of CHCl₃-MeOH—H₂O=15:3:1 as an elution solvent was used, and each of 20 mL of fraction was divided. Each divided fraction was confirmed by TLC (CHCl₃-MeOH—H₂O=10:3:1), and similar portions were collected and concentrated, and thus 14 fractions (MOE-1 to MOE-14) were obtained. Two single compounds, MOE-8 (astragalin, 106.4 mg) and MOE-11 (isoquercetin, 164.6 mg), were obtained (FIG. 1A).

1) astragalin (MOE-8)

Yellowish powder (MeOH); positive FAB/MS m/z 471 [M+Na]⁺; ¹H-NMR (600 MHz, CD₃OD, δ_{H}) 8.05 (2H, d, J=9.0 Hz, H-2′,6′), 6.90 (2H, d, J=9.0 Hz, H-3′,5′),

6.38 (1H, d, J=1.2 Hz, H-8), 6.20 (1H, d, J=1.2 Hz, H-6), 5.22 (1H, d, J=7.2 Hz, H-1″), 3.71 (1H, dd, J=12.0, 1.8 Hz, H-6″ a), 3.60 (1H, dd, J=12.0, 6.0 Hz, H-6″ b), 3.32-3.58 (4H, overlapped, H-2″-5″); ¹³C-NMR (150 MHz, CD₃OD, δC) 179.5 (C-4), 166.0 (C-7), 163.1 (C-5), 161.6 (C-4′), 159.2 (C-9), 158.5 (C-2), 135.6 (C-3), 132.4 (C-2′), 132.4 (C-6′), 122.8 (C-1′), 116.2 (C-3′), 116.2 (C-5′), 105.8 (C-10), 104.4 (C-1″), 100.0 (C-6), 94.9 (C-8), 78.4 (C-5″), 78.0 (C-3″), 75.8 (C-2″), 71.4 (C-4″), 62.7 (C-6″).

2) isoquercetin (MOE-11)

Yellowish powder (MeOH); positive FAB/MS m/z 487 [M+Na]⁺; ¹H-NMR (600 MHz, CD₃OD, δ_{H}) 7.74 (1H, d, J=1.8 Hz, H-2′), 7.58 (1H, dd, J=8.4, 1.8 Hz, H-6′), 6.88 (1H, d, J=8.4 Hz, H-5′), 6.35 (1H, d, J=1.2 Hz, H-8), 6.17 (1H, d, J=1.2 Hz, H-6), 5.18 (1H, d, J=7.2 Hz, H-1″), 3.76 (1H, dd, J=12.0, 1.8 Hz, H-6″a), 3.71 (1H, dd, J=12.0, 6.0 Hz, H-6″ b), 3.55-3.27 (4H, overlapped, H-2″-5″); ¹³C-NMR (150 MHz, CD₃OD, (5c) 179.5 (C-4), 166.1 (C-7), 163.0 (C-5), 159.2 (C-9), 158.5 (C-2), 150.0 (C-3′), 145.9 (C-4′), 135.8 (C-3), 123.4 (C-1′), 123.1 (C-6′), 117.8 (C-5′), 116.2 (C-2′), 105.8 (C-10), 104.7 (C-1″), 100.1 (C-6), 95.0 (C-8), 78.4 (C-5″), 78.2 (C-3″), 75.9 (C-2″), 71.3 (C-4″), 62.7 (C-6″).

SiO₂ column chromatography was conducted on 4 g of EtOH fraction of the fermented Moringa extract. A column with size (y) 7×20 cm) was used, and the elution was performed with increasing polarity using CHCl₃-MeOH—H₂O (50:3:1→40:3:1→20:3:1→10:3:1, each 2.7 L) as an elution solvent. Each of 15 mL of fraction was divided. Each fraction was confirmed by TLC (CHCl₃-MeOH—H₂O=15:3:1), and similar portions were combined and concentrated, and thus 19 fractions (MOFE-1 to MOFE-19) were obtained. A single compound, MOFE-11 (niazirin, 33.5 mg), was obtained. The MOFE-8 fraction (210 mg, Ve/Vt=0.163-0.264) was subjected to ODS column chromatography (ϕ 3×7 cm, MeOH—H₂O=2:1→3:1, 0.6 L respectively), and thus seven fractions (MOFE-8-1 to MOFE-8-7) were obtained. MOFE-8-3 (kaempferol, 22.6 mg) was obtained as a single compound. The MOFE-16 fraction (47.7 mg, Ve/Vt=0.418-0.458) was subjected to ODS column chromatography (ϕ 3×10 cm, MeOH—H₂O=1:1→2:1, 0.6 L respectively), and thus nine fractions (MOFE-16-1 to MOFE-16-9) were obtained. Two single compounds, MOFE-16-5(6″-O-acetyl astragalin, 6.8 mg) and MOFE-16-7 (quercetin, 21.4 mg), were obtained. Consequently, four single compounds were obtained from EtOH fraction of the fermented Moringa extract (FIG. 1B).

1) niazirin (MOFE-11)

Yellowish crystal (MeOH); positive FAB/MS m/z 491 [M+H]⁺; ¹H-NMR (600 MHz, pyridine-cis, δ_{H}) 7.31 (2H, d, J=8.4 Hz, H-2,6), 7.23 (2H, d, J=8.4 Hz, H-3,5), 3.88 (2H, s, H-7), 6.01 (1H, m, H-1′) 4.66 (1H, m, H-2′), 4.62 (1H, dd, J=9.6, 3.0 Hz, H-3′), 3.26 (1H, m, H-4′), 3.48 (1H, dd, J=9.0, 9.0 Hz, H-5′), 1.54 (3H, d, J=6.0 Hz, H-6′); ¹³C-NMR (150 MHz, pyridine-d₅, δC) 156.9 (C-1) 135.7 (C-4), 129.7 (C-3), 129.7 (C-5), 124.6 (C-8), 119.4 (C-2) 117.5 (C-1′), 117.5 (C-4′), 73.7 (C-3′), 75.5 (C-2′), 71.9 (C-5′), 70.9 (C-7), 22.6 (C-6), 18.6 (C-6′).

2) kaempferol (MOFE-8-3)

Yellowish powder (CHCl₃); positive FAB/MS m/z 287 [M+H]⁺; ¹H-NMR (600 MHz, CD₃OD, δ_{H}) 8.10 (2H, d, J=8.4 Hz, H-2′,6′), 6.91 (2H, d, J=8.4 Hz, H-3′,5′), 6.38 (1H, s, H-8), 6.18 (1H, s, H-6); ¹³C-NMR (150 MHz, CD₃OD, δ_{C}) 177.3 (C-4), 165.5 (C-7), 162.5 (C-5), 160.5 (C-4′), 158.2 (C-9), 148.0 (C-2), 137.2 (C-3), 130.7 (C-2′), 130.7 (C-6′), 123.7 (C-1′), 116.3 (C-3′), 116.3 (C-5′), 104.5 (C-10), 99.3 (C-6), 94.5 (C-8).

3) 6″-O-acetyl astragalin (MOFE-16-5)

Yellowish powder (MeOH); positive FAB/MS m/z 513 [M+Na]⁺; ¹H-NMR (600 MHz, CD₃OD, δ_{H}) 8.02 (2H, d, J=9.0 Hz, H-2′,6′), 6.85 (2H, d, J=9.0 Hz, H-3′,5′),

6.40 (1H, d, J=1.2 Hz, H-8), 6.20 (1H, d, J=1.2 Hz, H-6), 5.15 (1H, d, J=7.2 Hz, H-1″), 4.16 (1H, dd, J=12.0, 1.8 Hz, H-6″a), 4.15 (1H, dd, J=12.0, 6.0 Hz, H-6″b), 3.43-3.26 (4H, overlapped, H-2″˜5″), 1.81 (3H, s, H-ace-2); ¹³C-NMR (150 MHz, CD₃OD, δ_{C}) 179.6 (C-4), 172.6 (C-ace-1), 166.3 (C-7), 163.0 (C-5), 161.7 (C-4′), 159.6 (C-9), 158.7 (C-2), 135.5 (C-3), 132.4 (C-2′), 132.4 (C-6′), 122.9 (C-1′), 116.1 (C-3′), 116.1 (C-5′), 105.8 (C-10), 104.4 (C-1″), 100.1 (C-6), 95.0 (C-8), 78.0 (C-3″), 75.8 (C-2″), 75.7 (C-5″), 71.5 (C-4″), 64.4 (C-6″), 20.6 (C-ace-2).

4) quercetin (MOFE-16-7)

Yellowish powder (CHCl₃); positive FAB/MS m/z 301 [M+H]⁺; ¹H-NMR (600 MHz, CD₃OD, δ_{H}) 7.73 (1H, d, J=1.8 Hz, H-2′), 7.63 (1H, dd, J=8.4, 1.8 Hz, H-6′), 6.88 (1H, d, J=8.4 Hz, H-5′), 6.38 (1H, s, H-8), 6.17 (1H, s, H-6); ¹³C-NMR (150 MHz, CD₃OD, od 179.4 (C-4), 166.1 (C-7), 163.2 (C-5), 159.2 (C-9), 158.7 (C-2), 150.2 (C-3′), 145.8 (C-4′), 135.8 (C-3), 123.4 (C-1′), 123.2 (C-6′), 117.8 (C-5′), 116.1 (C-2′), 105.7 (C-10), 100.0 (C-6), 94.9 (C-8).

### Example 3. Evaluation of Inhibitory Efficacy of Adipocyte Differentiation

The inhibitory efficacy on adipocyte differentiation in Moringa powder and fermented Moringa extract obtained from Example 1 was measured.

Preadipocytes (3T3-L1) from mice were purchased from ATCC (American Type Culture Collection). DMEM culture medium, Trypsin/EDTA, FBS (fetal bovine serum), and BCS (Bovine calf serum) were purchased from Gibco (Invitrogen corporation). AdipoRed assay reagent for measurement was purchased from Cambrex.

Specifically, 3T3-L1 preadipocytes were cultured in DMEM medium containing 10% BCS and antibiotics, and maintained in a CO 2 incubator with 5% carbon dioxide supply. The cells were seeded at a concentration of 1×10⁶ cells in a 96-well plate and cultured until they reached 100% confluency. To measure adipocyte differentiation, the cells were treated with MD medium (0.5 mM 3-isobutyl-1-methylxanthine and 1 μM dexamethasone). After 2 days, the medium was replaced with DMEM medium containing 10% FBS, and further cultured for 6 days. Moringa powder and fermented Moringa extract were treated at various concentrations.

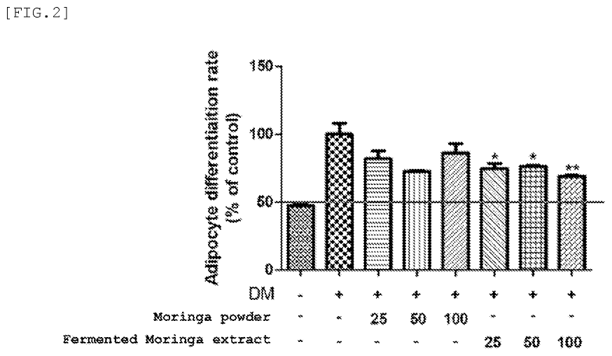

Furthermore, after adipocytes were differentiated and cultured for 8 days, the adipocytes were fixed with a 4% formaldehyde solution at room temperature for 5 hours, and then washed with a phosphate buffer and treated with the AdipoRed assay reagent as a fat measurement reagent. After 10 minutes, fluorescence intensity was measured using a fluorescence intensity reader. The results obtained through the aforementioned experimental method are presented in FIG. 2.

As shown in FIG. 2, both Moringa powder and fermented Moringa extract inhibited the adipocytes differentiation compared to a positive control group, Differentiation Medium (DM). Additionally, when comparing Moringa powder and fermented Moringa extract at each concentration, the fermented Moringa extract exhibited a lower adipocyte differentiation rate than Moringa powder at all concentrations. In particular, the fermented Moringa extract at a concentration of 25 μg/mL showed similar activity to the concentration of 50 μg/mL of Moringa powder.

Subsequently, the inhibitory effect on adipocyte differentiation in the six compounds isolated in Example 2 was measured. The experimental procedure was conducted in the same manner as described above.

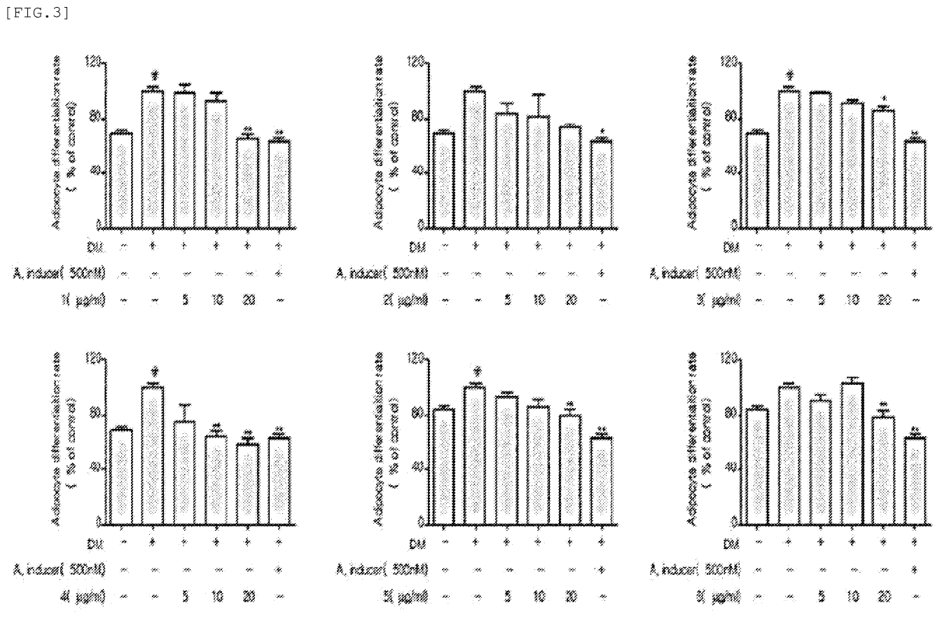

FIG. 3 represents the results of measuring the adipocyte differentiation rate of the compounds isolated from Moringa powder and fermented Moringa extract (1, isoquercetin; 2, astragalin; 3, kaempferol; 4, 6″-O-acetyl astragalin; 5, niazirin; 6, quercetin).

As shown in FIG. 3, when evaluating the inhibition of adipocyte differentiation for the six active compounds isolated from Moringa powder and fermented Moringa extract, excellent adipocyte differentiation inhibition activity was observed for MOE-11 (isoquercetin) and MOFE-16-5 (6″-O-acetyl astragalin).

The results indicate that Moringa powder possesses an adipocyte differentiation inhibition effect, and in particular, fermented Moringa extract exhibits superior adipocyte differentiation inhibition compared to Moringa powder. The results suggest that fermented Moringa extract can be utilized as a composition for preventing and treating obesity.

### Example 4. Evaluation of Inhibitory Efficacy of Adipocyte Differentiation after Hepatic Metabolism

The Moringa powder and fermented Moringa extract obtained from Example 1 were evaluated for their efficacy in inhibiting adipocyte differentiation after hepatic metabolism, respectively.

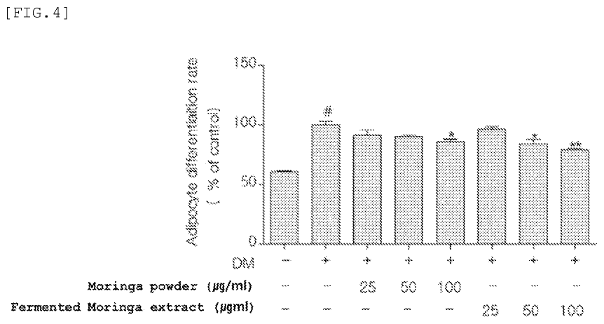

Hepa-1c1c7 mouse hepatocytes were treated with each sample at various concentrations and incubated for 24 hours in a 5% CO₂ incubator. The supernatant was then collected and used to treat 3T3-L1 mouse preadipocytes for 8 days to assess the inhibition of adipocyte differentiation. The results of the experiments conducted are presented in FIG. 4.

As shown in FIG. 4, both the Moringa powder and fermented Moringa extract decreased the adipocyte differentiation rate after hepatic metabolism. Furthermore, when comparing the Moringa powder and fermented Moringa extract at each concentration, the fermented Moringa extract exhibited a lower adipocyte differentiation rate than the Moringa powder at all concentrations.

The results indicate that Moringa powder has an ability to inhibit adipocyte differentiation after hepatic metabolism, and particularly, the fermented Moringa extract shows superior efficacy in inhibiting adipocyte differentiation after hepatic metabolism compared to Moringa powder. The results suggest that it can be utilized as a composition for the prevention and treatment of fatty liver disease, one of the metabolic disorders related to obesity.

As described above, while specific embodiments of the present invention have been detailed, a person skilled in the art, understanding the concept of the present invention, can easily propose other embodiments or regressive invention within the scope of the same concept by adding, modifying or deleting different components. Therefore, the embodiments described above should be understood as illustrative and not limiting in all aspects. The scope of the present invention is defined by the claims set forth below, and any modifications or variations derived from the meaning and scope of the claims and the equivalent concept are considered to be within the scope of the present invention.

### DEPOSIT NUMBER

Depositary Organization: Korean Culture Center of Microorganisms
Deposit Number: KCCM12830P
Deposit Date: Nov. 11, 2020

1. A pharmaceutical composition for preventing or treating obesity, comprising fermented Moringa (Moringa oleifera) extract as an active ingredient,
wherein the fermented Moringa extract is prepared by adding a strain of Monascus genus to Moringa powder and fermenting.

2. The composition of the claim 1, wherein the strain of Monascus genus is Monascus purpureus.

3. The composition of the claim 2, wherein the Monascus purpureus is Monascus purpureus SL-1 (Deposit Number KCCM12830P).

4. The composition of the claim 1, wherein the fermentation is performed at 30 to 40° C. for 1 to 5 days.

5. The composition of the claim 1, wherein the fermented Moringa extract is one or more selected from a group consisting of niazirin, kaempferol, 6″-O-acetyl astragalin and quercetin as an active ingredient.

6. The composition of the claim 1, wherein the composition inhibits differentiation of adipocytes.

7. A food composition for preventing or improving obesity, comprising fermented Moringa (Moringa oleifera) extract as an active ingredient,
wherein the fermented Moringa extract is prepared by adding a strain of Monascus genus to Moringa powder and fermenting.

8. A health functional food composition for preventing or improving obesity, comprising fermented Moringa (Moringa oleifera) extract as an active ingredient,
wherein the fermented Moringa extract is prepared by adding a strain of Monascus genus to Moringa powder and fermenting.

9. A method for preventing or treating obesity, comprising administering the composition selected from claim 1 to a subject.
